(19) United States
(12) Patent Application Publication
(10) Pub. No.: US 2006/0094045 A1
(43) Pub. Date: May 4, 2006

(54) Macrocyclic chelators for gene-silencing or gene disruption

(76) Inventors: Eddie Chang, Silver Spring, MD (US); Andy Knight, New Orleans, LA (US); Ellen Goldman, Germantown, MD (US); James Deleilanty, Arlington, VA (US); Rajeev Soni, New Dehli (IN)

Correspondence Address: NAVAL RESEARCH LABORATORY;ASSOCIATE COUNSEL (PATENTS), CODE 1008.2, 4555 OVERLOOK AVENUE, S.W., WASHINGTON, DC, 20375-5320, US

(21) Appl. No.: 11/251,542

(22) Filed: Sep. 30, 2005

Related U.S. Application Data
(60) Provisional application No. 60/624,495, filed on Nov. 1, 2004.

(51) Int. Cl. C12Q 1/68; C07F 15/06; C07H 21/04

(52) U.S. Cl. 435/6; 536/25.32; 540/465

(57) ABSTRACT

A composition comprising a kinetically inert ion coordinated to a macrocyclic chelator or ligand, a first functional group bonded to the macrocyclic chelator or ligand, an oligonucleotide, and a second functional group bonded to the oligonucleotide, wherein the first functional group covalently bonds to the second functional group.

This application claims the benefit of the earlier filed provisional application U.S. Ser. No. 60/624,495 filed on Nov. 1, 2004, the contents of which are herein incorporated by reference.

### BACKGROUND

1. Field of the Invention

The present invention relates to macrocyclic chelators and more specifically to macrocyclic chelators for gene-silencing or gene disruption.

2. Description of the Related Art

There has been much interest in the development of small metal complexes that can efficiently catalyze the hydrolysis of DNA and RNA for the purpose of making sequence-specific systems that can be used for gene-cloning, gene-mapping, or therapeutics. See, e.g., Chin, J. Acc. Chem. Res. 1991, 24, 145-152; Kim, N.; Suh, J. J. Org. Chem. 1994, 59, 1561-1571; Williams, N. H.; Chin, J. Chem. Commun. 1996, 131-132; Williams, N.; Takasaki, B.; Wall, M.; Chin, J. Acc. Chem. Res. 1999, 32, 485-493; Bashkin, J. K.; Sampath, U.; Frolova, E. Appl. Biochem. Biotechnol. 1995, 54, 43-56; Komiyama, M.; Sumaoka, J. Current Opinion in Chemical Biology 1998, 2, 751-757; Hegg, E. L.; Burstyn, J. N. Coord. Chem. Rev. 1998, 173, 133-165; Bashkin, J. K. Curr. Opin. Chem. Biol. 1999, 3, 752-758; and Komiyama, M.; Takeda, N.; Shigekawa, H. Chem. Commun. 1999, 1443-1451, the entire contents of each are incorporated herein by reference.

The general approach to suppression of protein translation would be to inhibit the ability of either DNA or RNA to act as templates for transcription and translation, respectively. This can be done either by blocking the site of transcription/translation (antisense) or by hydrolyzing those sites (RNAi). The common view of the function of a chemical “artificial nuclease” is that it must be able to attack and breakdown the very stable phosphodiester bonds of nucleic acids. It would be of further advantage if these systems were to possess an ability to recognize specific sequences of nucleotides and thereby hydrolyze only (or mostly) the nearby phosphodiester bond of a target sequence. Metal-chelator complexes, with sequence-specificity derived from attaching a recognition element to the hydrolytic center, embody the functionalities of an artificial nuclease. Thus, a full sequence-recognizing, artificial nuclease can be thought of consisting of three components: a hydrolytic end, such as a metal-chelating complex; a linker between the metal complex and a sequence-recognition end; and the controlled targeting (recognition functionality) component of the “chemical nuclease”.

It has been estimated that the half-life for the hydrolysis of the bond, at 25° C. and neutral pH, varies between 130,000 years (Radzicka, A.; Wolfenden, R. Science 1995, 267, 90-93, the entire contents of which is incorporated herein by reference) to tens or hundreds of billions of years (Williams, N.; Takasaki, B.; Wall, M.; Chin, J. Acc. Chem. Res. 1999, 32, 485-493, the entire contents of which is incorporated herein by reference) (c.f., the age of earth is around 4 billion years). Obviously, though, there are ways to break more easily the phosphodiester bonds, as for example, nucleases, with their half-lives of minutes, are among the most efficient hydrolases known (Catalog, N. E. B. 2000).

These impressive rates are achieved by lowering or favoring the transition state energy structure(s) through bond strains, entropic mechanisms, and other multiple enzyme-substrate interactions (Hegg, E. L.; Burstyn, J. N. Coord. Chem. Rev. 1998, 173, 133-165, the entire contents of which is incorporated herein by reference). However, not all the design strategies used by enzymes can be incorporated into small artificial nuclease systems.

Previous efforts by others have shown the feasibility of man-made catalytic degradation of the DNA bond of through oxidative-degradation (Chen, C. H. B.; Milne, L.; Landgraf, R.; Perrin, D. M.; Sigman, D. S. Chembiochem 2001, 2, 735-740; Perrin, D. M.; Mazumder, A.; Sigman, D. S. In Progress in Nucleic Acid Research and Molecular Biology, Vol 52, 1996; Vol. 52, pp 123-151; Sigman, D. S.; Chen, C. H. B. Annu. Rev. Biochem. 1990, 59, 207-236, the entire contents of each are incorporated herein by reference), use of labile transition-metal ion chelator complexes (Hegg, E. L.; Burstyn, J. N. Inorg. Chem. 1996, 35, 7474-7481; Sumaoka, J.; Kawata, K.; Komiyama, M. Chemistry Letters 1999, 439-440; Yashiro, M.; Ishikubo, A.; Komiyama, M. Abstr. Pap. Am. Chem. Soc. 1996, 212, 303-INOR, the entire contents of each are incorporated herein by reference), and chelator-lanthanide ions (Kuzuya, A.; Komiyama, M. Chemistry Letters 2000, 1378-1379; Komiyama, M.; Takeda, N.; Shigekawa, H. Chem. Commun. 1999, 1443-1451; Sumaoka, J.; Kajimura, A.; Imai, T.; Ohno, M.; Komiyama, M. Nucleosides Nucleotides 1998, 17, 613-623, the entire contents of each are incorporated herein by reference). There were advantages and problems with each of these systems: Oxidative-degradation was fast but required peroxide and was non-localized and non-specific, breaking down all bonds in the vicinity through a variety of mechanisms. The labile metal-ions, such as Cu²⁺ or Zn²⁺, were catalytically active, but relatively weak. Lanthanide ions are potent hydrolytic agents, but stable complexes in solution were difficult to obtain.

Co(III), with its high charge-density, is a potent candidate for use in hydrolyzing phosphodiesters, for example, recently published values for DNA cleavage show that Co(III) complexes have rate constants around 2×10⁻⁴ s⁻¹, which is about three fold higher than that for europium(III) salts. Hettich, R.; Schneider, H. J. J. Am. Chem. Soc. 1997, 119, 5638-5647, the entire contents of which is incorporated herein by reference. Although Co(III) is not stable by itself, in aqueous solutions, it can be stabilized by coordinating with donor atoms (usually N) that make strong contributions to the ligand field. These Co(III)-chelator complexes have been used for mechanistic studies of phosphodiester cleavage for both its efficient hydrolysis rates and kinetic inertness. That is, the complexes promote fast hydrolysis of the phosphodiester bond but are kinetically “slow” in letting go of the hydrolyzed phosphate. Douglas, B. E.; McDaniel, D. H.; Alexander, J. J. Concepts and Models of Inorganic Chemistry 2md Edition; J. Wiley & Sons, Inc.: New York, 1983, the entire contents of which is incorporated herein by reference. The kinetic inertness (“stickiness”) of the Co(III) may be overcome (i.e., at elevated temperatures) but, for gene-silencing, this property is an added advantage for disruption of gene function.

Tetramine chelators (see FIG. 1), such as cyclen (1,4,7,10-tetraazacyclododecane) or trpn (tris(3-aminopropyl)amine), have the ability to spontaneously chelate Co(III) in the cis configuration and have shown some of the highest hydrolysis rates for these Co(III) complexes. Another advantage of these tetramine chelators is the ability to attach functional groups to the amines such as, for example, polynucleotides.

Coupling of Co(III)-Chelator to Polynucleotides: Polyamine chelators, in principle, can easily be linked to other groups through the amines. Brinkley, M. Bioconjugate Chem. 1992, 3, 2-13; and Mattson, G.; Conklin, E.; Desai, S.; Nielander, G.; Savage, M. D.; Morgensen, S. Mol. Biol. Rep. 1993, 17, 167-183, the entire contents of both are incorporated herein by reference. There are standard chemical approaches for coupling to the amines, such as using carbonyldiimidazole or carbodiimides to join the 5′-phosphate terminal of an oligonucleotide to low molecular-weight amines. Komiyama, M.; Takeda, N.; Shiiba, T.; Takahashi, Y.; Matsumoto, Y.; Yashiro, M. Nucleosides Nucleotides 1994, 13, 1297-1309; and Milne, L.; Xu, Y.; Perrin, D. M.; Sigman, D. S. Proc NAT ACAD SCI USA 2000, 97, 3136-3141, the entire contents of both are incorporated herein by reference. These reactions are generally done in organic solvents in the absence of transition metal ions. For cases where water-solubility is an issue or the amines are only weakly nucleophilic, there are more specialized coupling and activating reagents available. Sometimes, a balance has to be struck between ease of coupling and the method by which Co(III) is introduced into the chelators (see Design of Hybridization below).

Controlled Targeting by Artificial Nuclease: It has been demonstrated that metal-ion complexes coupled to single-strand nucleic acids will recognize its targeted nucleic sequences and cleave the DNA with specificity. Chen, C. H. B.; Milne, L.; Landgraf, R.; Perrin, D. M.; Sigman, D. S. Chembiochem 2001, 2, 735-740; Perrin, D. M.; Mazumder, A.; Sigman, D. S. In Progress in Nucleic Acid Research and Molecular Biology, Vol 52, 1996; Vol. 52, pp 123-151; Sigman, D. S.; Chen, C. H. B. Annu. Rev. Biochem. 1990, 59, 207-236; Kuzuya, A.; Komiyama, M. Chemistry Letters 2000, 1378-1379; Komiyama, M.; Takeda, N.; Shigekawa, H. Chem. Commun. 1999, 1443-1451; and Sumaoka, J.; Kajimura, A.; Imai, T.; Ohno, M.; Komiyama, M. Nucleosides Nucleotides 1998, 17, 613-623, the entire contents of each are incorporated herein by reference. However, for purposes of decontamination via antisense, it will not be necessary to fully digest a DNA or RNA strand to deactivate an organism, as long as the critical sites remain permanently blocked. The strong affinity of the proposed Co(III) complexes of the present invention for the bases and the phosphate groups of the nucleotides, therefore, is an advantage to the general goal of deactivating genetic materials. The molecular recognition potential of the chemical nucleases, furthermore, means that these chemical nucleases can potentially be used in a novel approach to interdict whole classes of organisms (e.g., the filoviruses).

Relation to Antisense/RNAi Technology: The binding step of these artificial nucleases resembles, to an extent, the standard anti-sense approach to block the production of critical proteins in the standard DNA to RNA to protein paradigm. Co(III) systems have significant advantages in this regard because not only does it bind strongly to nucleotides but it also helps sever the nucleotides' phosphodiester bonds to permanently disable the translation mechanism.

This is an important advantage because standard antisense approaches involve the synthesis of oligodeoxynucleotides (ODN's) that bind to their complementary sequences on the mRNA, thereby blocking translation and inhibiting the production of the target protein. However, binding to the RNA is often not stable. Ribosomes can effectively compete with the oligonucleotides to bind with the RNA and consequently ensure continuous production of the target protein. The competitive edge of ribosomes is facilitated by their intrinsic “unwindase” activity, which allows them to read tangled messages, thus overcoming the effect of the antisense ODN.

The preferred solution to this problem relies on the ability of the antisense oligonucleotides to employ the enzyme RNase H. Bonham, M. A.; Brown, S.; Boyd, A. L.; Brown, P. H.; Bruckenstein, D. A.; Hanvey, J. C.; Thomson, S. A.; Pipe, A.; Hassman, F.; Bisi, J. E. e. a. Nucleic Acids Res. 1995, 23, 1197-1203; Larrouy, B.; Blonski, C.; Boiziau, C.; Stuer, M.; Moreau, S.; Shire, D.; Toulme, J. J. Gene 1992, 121, 189-194; and Walder, R. Y.; Walder, J. A. Proceedings of the National Academy of Sciences, USA 1988, 85, 5011-5015, the entire contents of each are incorporated herein by reference. RNase H recognizes the DNA:RNA duplex and acts as a DNA dependent RNA hydrolysis catalyst. This degrades the RNA leaving the antisense ODN free to bind to other mRNA molecules and repeating the hydrolysis cycle. However, RNase H itself poses a drawback because duplexes as short as 5 base pairs are cleaved by RNase H leading to poor specificity of the antisense ODN's (Crouch, R.; Dirksen, M. In Nucleases; Linn, S. a. R., R., Ed.; Cold Spring Harbor Laboratory Press: Cold Spring Harbor, pp 211-241, the entire contents of which are incorporated herein by reference).

There are also other issues that have hindered the efficient exploitation of using simple ODN's. One important concern is the stability of the antisense ODN. Stein, C. A. Leukemia 1992, 6, 967-974; Stein, C. A.; Cheng, Y. C. Science 1993, 261, 1004-1012; Stein, C. A.; Narayanan, R. Current Opinion in Oncology 1994, 6, 587-594; and Alama, A.; Barbieri, F.; Cagnoli, M.; Schettini, G. Pharmacological Research 1997, 36, 171-178, the entire contents of each are incorporated herein by reference. Conventional ODN's are prone to nuclease degradation inside the cell. Modified ODN's—have been synthesized with different backbones to improve their stability but they either fail to recruit RNase H or exhibit non-sequence specificity. A classical example of stable antisense oligos are S-DNA (phosphorothioate) oligos that possess enhanced stability but exhibit low sequence specificity because of their weak binding. Stein, C.; Cohen, J. In Oligodeoxynucleotides: Antisense inhibitors of gene expression.; Cohen, J., Ed.; CRC Press, Inc.: Boca Raton, Fla., pp 97-117; Krieg, A.; C., S. Antisense Research and Development 1995, 5, 241; Stein, C. A. Trends in Biotechnology 1996, 14, 147-149.; and Agrawal, S.; Zhao, Q. Current Opinion in Chemical biology 1998, 2, 519-528, the entire contents of each are incorporated herein by reference. In addition, they promiscuously bind cellular protein molecules thus losing their effectiveness as antisense agents.

Peptide nucleic acids, in which the backbone consists of N-(2-aminoethyl)-glycine units linked by peptide bonds instead of a sugar and phosphate groups, have gained considerable importance by virtue of their being nuclease resistant and their ability to form stable complexes with nucleic acids. Larsen, H. J.; Bentin, T.; Nielsen, P. E. Biochimica et Biophysica Acta 1999, 1489, 159-166; Ray, A.; Norden, B. Faseb J. 2000, 14, 1041-1060; and Nielsen, P. E. Pharmacology Toxicology 2000, 86, 3-7, the entire contents of each are incorporated herein by reference. More recently, morpholino oligos have also been reported to afford high efficacy, specificity, and resistance to nucleases. Summerton, J.; Stein, D.; Huang, S. B.; Matthews, P.; Weller, D.; Partridge, M. Antisense Nucleic Acid Drug Development 1997, 7, 63-70; Summerton, J.; Weller, D. Antisense Nucleic Acid Drug Development 1997, 7, 187-195; and Summerton, J. Biochimica et Biophysica Acta 1999, 1489, 141-158, the entire contents of each is incorporated herein by reference. However, they fail to recruit RNase H relying only on the binding specificity of the oligo.

The major difficulties with RNAi lie with the lack of a reliable method of targeting and delivery of dsRNA. Issues such as activation of the cells' antiviral defense mechanism by long strand dsRNA, identification of a viable target region, and uncontrolled global changes in gene expression of cells when dsRNA strands are introduced into the cells complicate any potential RNAi applications using short, interfering RNA (siRNA). While activation of cellular antiviral mechanisms may be desirable for antiviral applications, most viruses can shut down the defenses once entry is achieved. Therefore, this viral defense strategy can be an impediment for siRNA therapeutics.

Since only short RNA strands can be used for siRNA, specificity of the target can be an issue. Additionally, determining which sequences will work for siRNA still remains a problem to be solved for each target gene. Therefore, multiple regions are generally screened for each target. The screening requires using either synthetic RNAs, which are expensive, or with cloned DNA sequences, which is time consuming.

The current alternatives for decontamination involve harsh chemicals such as peroxides or bleach. These chemicals can be harmful to the surfaces, such as sensitive equipment surfaces, being decontaminated. Antisense and RNAi approaches may also be considered alternatives, especially for biomedical applications, but these technologies have the shortcomings discussed above.

### SUMMARY

The approach of the present invention has some similarities to the general antisense technologies that have been developed and to the newer RNA interference (RNAi) approach; but there are some crucial differences. For antisense to function, the antisense nucleotide must bind tightly to the target sense-strand. However, the binding of an antisense strand within the cell is a difficult proposition due to the “unwindase”-like activity of the ribosomes themselves, which can quite effective compete for binding to the target RNA strand. The present invention, on the other hand, does not just rely on hybridization to the target region—whether RNA or DNA. The macrocyclic chelator can bind tightly to the target (i.e., RNA) thus inhibiting translation. In addition, the macrocyclic chelator of the present invention actually seeks to hydrolyze the phosphodiester bonds, so the ribosome machinery can no longer act on the target gene. In the case of RNAi, the small-interfering RNA (siRNA) that is the active agent of the technology is exactly that, a short duplex strand of nucleotides, no more than about 20 nucleotides in original length and much shorter after cellular degradation. This length will limit the specificity with which the target regions can be tuned. Furthermore, the activity of the siRNA is not well understood at present—not all sequences will work and it is still a hit-and-miss proposition to find an active sequence. In the present invention, by using a combination of hybridizing nucleotides, macrocyclic chelator-nucleic acid interaction and catalysts, multiple pathways are used to obtain the “gene-silencing” effect.

### BRIEF DESCRIPTION OF THE DRAWINGS

These and other objects, features, and advantages of the invention, as well as the invention itself, will become better understood by reference to the following detailed description, appended claims, and accompanying drawings where:

FIG. 1 shows the structure for tetramine chelators cyclen (1,4,7,10-tetraazacyclododecane) and trpn (tris(3-aminopropyl)amine), which have the ability to spontaneously chelate Co(III) in the cis configuration and have shown some of the highest hydrolysis rates for these Co(III) complexes;

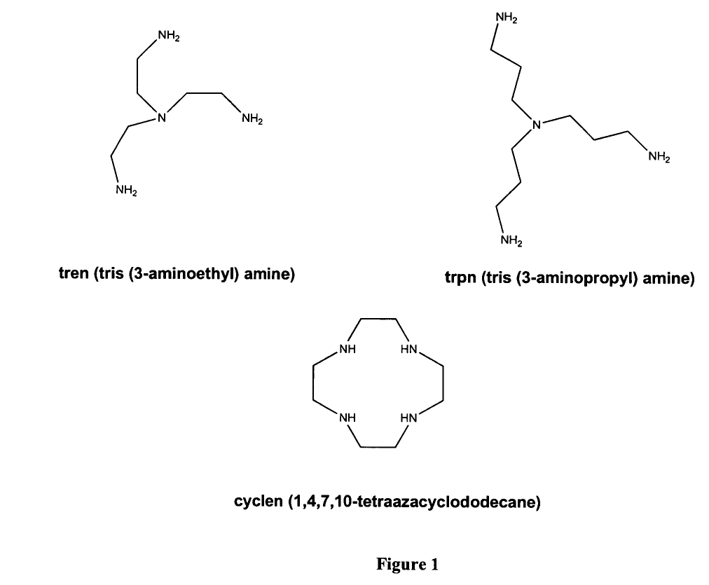

FIG. 2 shows that between pH 8.2 and 7.3, complexes 1 and 2 should convert from predominantly the dihydroxo to the aquo-hydroxo form;

FIG. 3 shows DNA digested with cocymba and cocycmmb at three pH conditions;

FIG. 4 shows the absence of the protein band in the Western blot as a function of cocycmmb concentration and also as a function of time of contact between Cocycmmb and the RNA; and

FIG. 5 shows an analysis by electrophoresis.

### DETAILED DESCRIPTION

Small macrocyclic-chelators are used either to prevent RNA from being translated into protein (gene silencing) or as anti-viral, anti-bacterial agents. Unlike other proposed metal-chelator systems for the sequence-specific hydrolysis of DNA or RNA that make use of the known ability of these metal-chelator complexes to hydrolyze the phosphodiester bonds of viral/bacterial nucleic acids, the present invention makes use of properties, such as, but not limited to, kinetic inertness (with the right choice of metal ion) and polyamine-nucleotide interactions (with appropriate choice of chelators). These systems can be used by themselves for non-specific gene silencing or for general viral and bacterial decontamination, e.g., of sensitive equipment surfaces. In addition, when nucleotide-binding groups, such as hybridization-capable oligonucleotides, or other nucleotide-binding groups (e.g., peptide nucleic acids, modified nucleic acids, triple-helix formers, or nucleotide-binding proteins and drugs), are attached to these complexes, the resulting system will be able to silence specific genes or attack specific viruses and bacteria. Thus, under these circumstances, the systems can behave like “artificial nucleases”, except they can recognize much longer nucleotide sequences and are much more stable. Therefore, these nuclease-like complexes can be used for both non-sequence-specific and sequence-specific inhibition of transcription/translation of viral/bacterial proteins, as well as other molecular biology tasks. Further, it is possible to engineer-in properties, such as “stickiness”, into the system so that it does not easily release the nucleic acid. The binding and blocking of translation is similar in concept to “antisense” and RNAi technologies, but this approach includes not only binding, but can also include destruction of the target sequence.

Preparation of Chelator-Metal Complexes

The preparation of Co(III)-cyclen complexes first involves the conversion of Co(II)Cl₂ to a stabilized Co(III) complex and then reacting the Co(III) complex with cyclen.

4-(1,4,7,10-Tetraazacyclotetradec-1-yl)methylbenzoic acid tetrahydrochloride (cycmba.4HCl, 1): A 250 mL flask was charged with cyclen (4.01 g, 23.3 mmol) ethanol (60 mL), water (13 mL) and a stirrer bar. Then, a cold (0° C.) mixture of LiOH (0.49 g, 12 mmol), p-bromotoluic acid (1.00 g, 4.65 mmol), water (50 mL) and ethanol (15 mL) was added to the cyclen solution in one portion, with stirring. The reaction mixture was refluxed for 3.5 h, allowed to cool to room temperature and concentrated to ca. 30 mL using rotary evaporation. The resulting solution was extracted with CHCl₃ until no more cyclen remained in the CHCl₃ washings (ca. 1000 mL). The aqueous phase was treated drop-wise with conc. HCl (ca. 7 mL) with stirring followed by ethanol (10 mL). The resulting white precipitate was collected by filtration, washed with ethanol and ether (50 mL) and dried in air to give 1 (see Scheme 1) as a hygroscopic white powder (0.49 g, 1.1 mmol, 24%). Anal. Calcd for C₁₆H₃₀Cl₄N₄O₂: C, 42.40; H, 6.89; N, 12.36. Found: C, 42.10; H, 6.92; N, 13.02. IR (KBr pellet): ν(C═O) 1730 cm⁻¹. ¹H NMR (D₂O): δ8.07 (d, ³J=8.8 Hz, 2H, C₆H₄), _{—}7.52 (d, ³J=8.4 Hz, 2H, C₆H₄), 3.92 (s, 2H, C₆H₄CH₂), 3.25-2.89 (m, 19H of 8CH₂, 3NH).

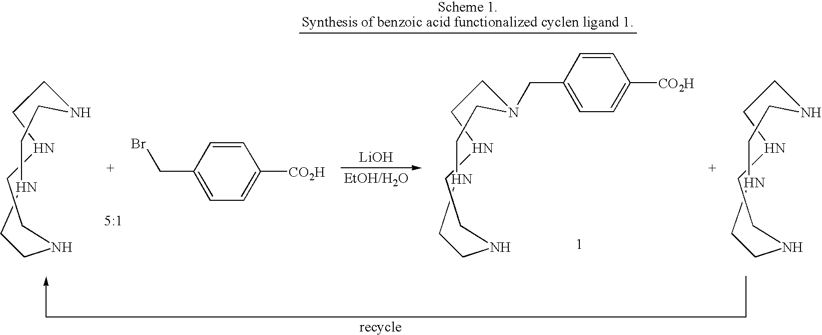

[Co(cycmba)Cl₂]Cl.1.5H₂O: Method A: A 100 mL flask was charged with cycmba.4HCl (0.41 g, 0.91 mmol), trisodium tris(carbonato)cobaltate(III) trihydrate (0.285 g, 0.775 mmol), and a stirrer bar. Then methanol (12 mL) and water (17 mL) were added and the mixture was refluxed for 4 hours. The red-purple solution was filtered hot, through a plug of Celite on a coarse frit and the resulting solution treated drop-wise with conc. HCl (3 mL). The solution was concentrated by rotary evaporation to ca. 10 mL and cooled in an ice-bath overnight. The resulting precipitate was collected by filtration, washed with 1:1 conc. HCl/H₂O (ca. 5 mL) methanol (ca. 5 mL) ether (ca. 10 mL) and dried in air to give [Co(cycmba)Cl₂]Cl.1.5H₂O as a violet solid. (0.221 g, 0.443 mmol, 57%). Anal. Calcd for C₁₆H₂₉Cl₃CoN₄O_{3.5}: C, 38.54; H, 5.86. Found: C, 38.32; H, 5.93.

Method B: A 500 mL flask was charged with cyclen (3.0 g, 17 mmol), acetonitrile (250 mL) and triethylamine (6 mL). Then, a solution of p-bromotolunitrile (3.33 g, 17.0 mmol) in acetonitrile (50 mL) was added dropwise with stirring over a period of 45 mins. The solution was refluxed for 4 hours, and reduced under rotary evaporation to give a yellow oil. CH₂Cl₂ (180 mL) was added and the resulting white precipitate of [Et₃NH]⁺Br⁻ was removed via filtration. The solution was concentrated to a yellow oil and a second volume of dichloromethane was added and the salt again removed via filtration. The solvent was removed under rotary evaporation to give 4-(1,4,7,10-tetraazacyclotetradec-1-yl)methylbenzonitrile (cycmbn) as a glassy yellow solid. Yield ˜4 g. IR (KBr pellet): ν(CN) 2227 cm⁻¹. ¹H NMR (CDCl₃): δ 7.8-7.4 (m, 4H, C₆H₄), 3.8 (s, 2H, C₆H₄CH₂), 3.6-2.4 (m, 19H of 8CH₂, 3NH). ¹H NMR indicated that the ligand was contaminated with [Et₃NH]⁺Br⁻ and was used without further purification. A portion of cycmba (0.75 g, 2.6 mmol) and cobalt chloride hexahydrate (0.62 g, 2.6 mmol) were dissolved in methanol (75 mL) and the solution was gently warmed for 30 mins. Then, hydrochloric acid (1 mL) was added drop-wise and the solution was stirred in air for 4 hours. The solution was concentrated to 15 mL via rotary evaporation and the resulting blue solid collected, washed with methanol and dried in air to give [Co(cycmbn)Cl₂]Cl as blue microcrystals (0.14 g, 0.31 mmol, 12% yield). The crystals were transferred to a 50 mL flask and concentrated hydrochloric acid (11 mL) was added drop-wise. The solution was refluxed for 35 hours, allowed to cool to room temperature, and the resulting violet microcrystals were collected via filtration, washed with water (5 mL) and acetonitrile (5 mL) and dried in air to give 2 (see Scheme 2) (0.10 g, 0.22 mmol, 71% yield). Anal. Calcd for C₁₆H₂₆Cl₃CoN₄O₂: C, 40.74; H, 5.56. Found: C, 40.53; H, 5.42. IR (KBr pellet): ν(OH) 3400 cm⁻¹, (br) ν(C═O) 1730 cm⁻¹. Electronic absorption spectra, λₘₐₓ, nm (ε, dm³ mol⁻¹ cm⁻¹): MeOH, 579 (310), 396 (347); H₂O, after 30 mins, 539 (427), 371 (326). ESI-MS (m/z): 363 ([Co(cycmba)]³⁺-2H), 399 ([Co(cycmba)Cl]²⁺-H), 435 ([Co(cycmba)Cl₂]⁺-H).

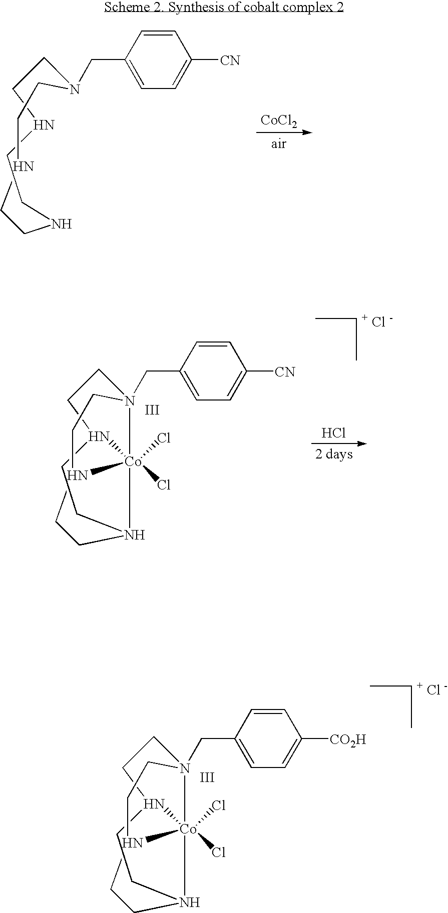

Dichloro{methyl 4-[(1,4,7,10-tetraazacyclododec-1-yl)methyl]-benzoate}cobalt(III) methylsulfate (CocycmmbCl₂-SO₄CH₃): The methyl ester of 2 was made as described previously. [Deschamps, 2003 #298] A 50 ml flask was charged with dichloro(4-[1,4,7,10-tetraazacyclotetradec-1-yl]methylbennzoic acid)cobalt(III) chloride (0.114 g, 0.242 mmol) and methanol (20 ml). Sulfuric acid (ca. 0.2 ml) was added dropwise, and the mixture was refluxed for 4 h. The deep blue-purple solution was allowed to cool to room temperature. After 12 h, purple crystals of the product (5) were collected by filtration, washed with ether and dried in air. After cooling the filtrate for 5 d at 255 K, a second crop of crystals was obtained. Yield 0.098 g, 72%.

Scheme 3 is for the conversion of 2 to 3. 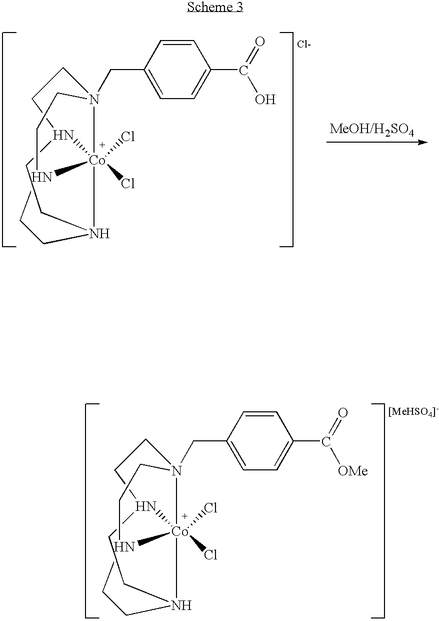
Coupling of Cylen-mba to Beads

Cyclen-MBA was coupled to functionalized diaminodipropylamine (DADPA) gel using the EDC/DADPA Immobilization Kit (Pierce Chemical, Rockford, Ill.). Briefly, 0.33 mL gel material was washed five times with 1 mL MilliQ water and the gel was resuspended in 1 mL DADPA conjugation buffer (0.1 M MES, 0.9% NaCl, pH 6.0). The gel was divided into 2 each 0.5 mL volumes (each containing 0.165 mL gel material).

In a 1.5 mL Eppendorf tube, the 0.165 mL gel (0.5 mL total volume containing 3 mmoles of available amino groups) was stirred. To the stirred gel, cyclen-MBA (4.6 mg (15 μmoles)) and EDC (144 mg (750 mmoles) were added. The reaction was allowed to proceed for 4 hours at room temperature. A control reaction was included in which cyclen-MBA was incubated with the gel material in the absence of EDC. The unreacted cyclen-MBA and EDC were removed by washing the gel 5 times with MilliQ water. See Scheme 4. 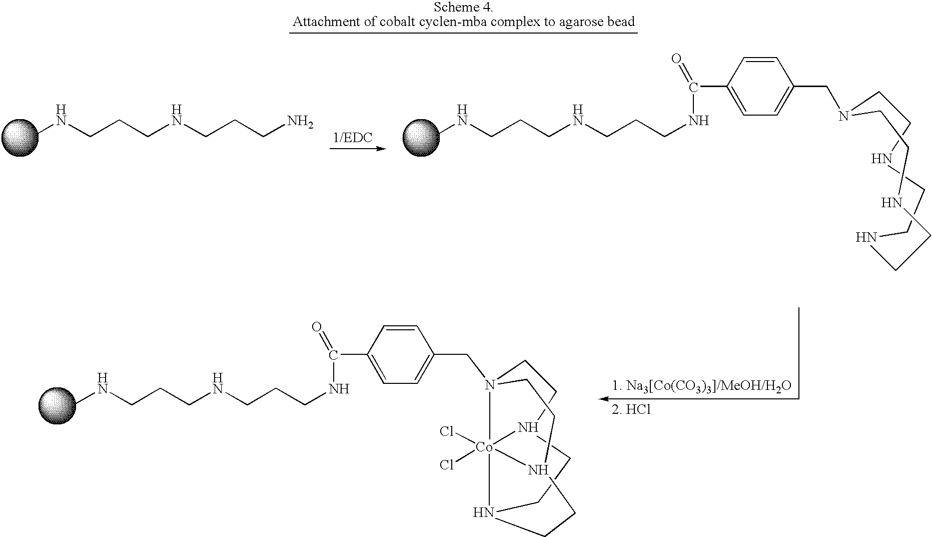
Coupling of Cyclen-Epoxide to Beads

Cyclen-epoxide was coupled to diaminodipropylamine (DADPA) gel as follows. Briefly, 0.055 mL of gel material (containing ˜0.5 mmole of available amino groups) was washed with MilliQ water as described above and resuspended in 0.1 mL coupling buffer (0.1 M sodium borate, pH 9.5). To the washed gel were added either 100 or 50 mmoles of cyclen-epoxide (in coupling buffer). Coupling reactions proceeded overnight at room temperature and the unreacted cyclen-epoxide was removed as above.

Coupling of ODN's to Cyclen-mba

Mix the cyclen-mba with amine-terminated ODN in 2 mL buffer A (15-mL screw cap tube). Make EDC/NHS coupling solution and use 3 mL immediately. Final concentrations: COOH 1 mM, NH₂ 5 mM, EDC 50 mM, NHS 3-5 mM. Rotate tubes at RT for 12 h (or at 4° C. for 24 h). Wash extensively with buffer B. Re-suspend in buffer A.

Western Blot Analysis of Cocycmmb Effect on Protein Translation:

Dose-Dependence

Luciferase control-RNA (at a final concentration of 0.4 ug/mL) was incubated with increasing concentrations of Cocycmmb for 24 h in 1×HBS buffer (pH 7.4) for 24 h. After the incubation period, the RNA template was translated into luciferase protein using the Rabbit Reticulocyte Lysate translation system (Promega) according to the manufacturer's instructions. After translation, the reaction was analyzed by reducing SDS-PAGE and western blotting to monitor the presence of the 60 kDa luciferase protein product. An aliquot (3 uL) of the translation reaction was run on a 10% NuPAGE Bis-Tris polyacrylamide gel under reducing conditions and transferred to a solid support membrane (PVDF). The membrane was blocked for 1 h in TBS-T (TBS containing 0.05% Tween) and subsequently incubated for 1 h with a 1:2500 dilution of a streptavidin-alkaline phosphatase conjugate. Detection was achieved by exposing the membrane to Western Blue substrate for alkaline phosphatase.

Time-Dependence

RNA incubations were done as described above except that the Cocycmmb concentration was held constant at 5 mM (and, more recently, down to 0.05 mM) and incubated for varying amounts of time that spanned 24 h. SDS-PAGE and Western blotting were done as described above.

Hydrolysis of BNPP by Cyclen-mba

The hydrolysis of BNPP by both Cocyc and 2 at 50° C., following activation using sodium hydroxide, has been measured (see Table 1). The second-order rate constants at pH 8.2 for both cyclen and 2 were similar at 14 and 12 M⁻¹ min⁻¹, respectively. The rate increased at pH 7.3 to a value of 19 M⁻¹ min⁻¹ for cyclen as compared to the published value of 2.8 M⁻¹ min⁻¹ at pH 7 (Chin, J.; Banaszczyk, M.; Jubian, V.; Zou, X. J. Am. Chem. Soc. 1989, 111, 186-190, the entire contents of which is incorporated herein by reference). The difference between the published value and ours can be attributed to different experimental conditions, such as buffer, pH, and the ratio of catalyst to substrate.

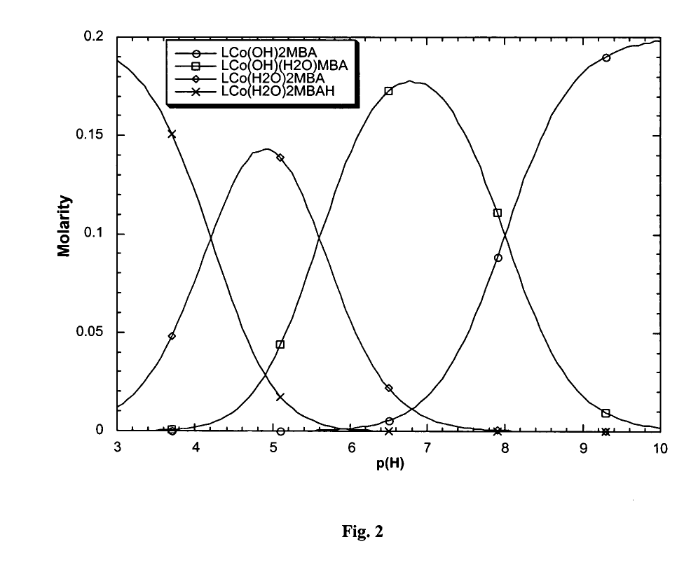

The much lower hydrolysis rate shown at pH 7.3 for Cocycmba is surprising. In order to determine whether the differences observed were mainly due to the carboxylic acid or to the presence of the benzene ring, the kinetics of 3 were measured at pH 7.3 and found to be 14 M⁻¹ min⁻¹, which is much closer to the Cocyc rate. Methylation of the benzoic acid takes away both the negative charge and an ability to effectively hydrogen bond. Therefore the results imply that the behavior of 2 can be mostly ascribed to the presence of the additional charge and the potential for hydrogen bonding. For example, the acid dissociation constants of cyclen of 5.6 and 8.0 mean that between pH 8.2 and 7.3 complexes 1 and 2 should convert from predominantly the dihydroxo to the aquo-hydroxo form (see FIG. 2). Since the benzoic acid is in the benzoate form (pKa 4.2), the increasing net positive charge at pH 7.3 of the coordination site may allow the benzoate to approach closer to Co(III) and interfere with the hydrolysis of BNPP.

TABLE 1
2^{nd} Order Rate Constants as a Function of pH

| | Cocyc | | |
|---|---|---|---|
| pH | (M⁻¹min⁻¹) | Cocycmba(M⁻¹min⁻¹) | Cocycmmb(M⁻¹min⁻¹) |
| 7.3 | 19 | 2.7 | 14 |
| 8.2 | 14 | 12 | 11 |

Hydrolysis of DNA by Cyclen-mba

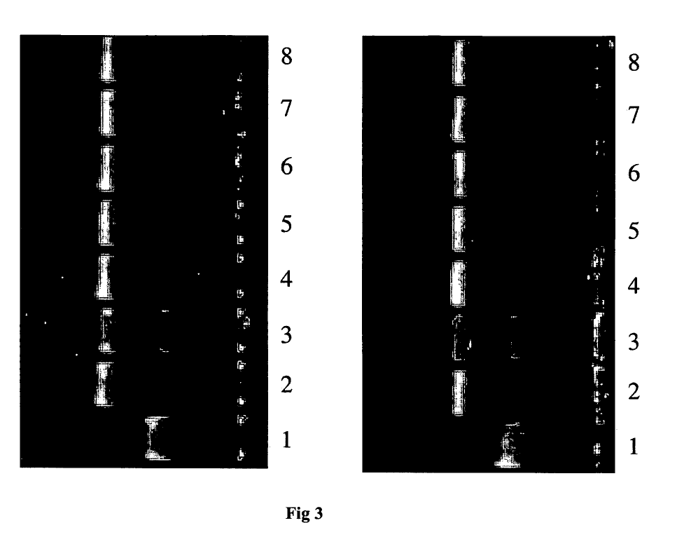

DNA was digested with cocycmba and cocycmmb at three pH conditions (FIG. 3). Hydrolysis of the phosophodiester bonds of the double stranded (ds) plasmid DNA should lead to a conversion from the covalently closed supercoiled form to the nicked form of the plasmid. Both cobalt catalysts showed the most nicking of DNA at pH 7 with lower levels of nicked DNA produced at pH 8 and 10. Plasmid DNA digested with a nicking enzyme (lane 1 FIG. 3) confirms that the slower moving band observed in digests with the cobalt catalysts is the nicked form of the plasmid. A faint band running slightly slower than the covalently closed supercoiled plasmid is probably linear DNA, and does not reduce in intensity on addition of the nickase. The cocycmmb appears to be a more effective catalyst than the cocycmba. Using NIH Image, it was found that under the conditions at pH 7, digestion with cocycmmb produced more nicked product (43%) than treatment with the cocycmba (25%). In comparison, treatment with the nickase enzyme resulted in 88% nicked product.

Although slower than the nickase enzyme, the cocycmmb is approaching the nickase efficiency producing about half the amount of nicked product as the enzyme. The cocycmmb results are instructive in that it more closely resembles a coupled complex than cocycmba. As with the hydrolysis of BNPP at neutral pH, the carboxylic acid functionality of the cocycmba is probably responsible for the difference in DNA hydrolysis between the two compounds.

FIG. 3. shows the following: a) 5 mM cocycmba and 1.19 μg (0.168 mM bp) pBluescript plasmid DNA in 0.1M MOPS buffer at pH 7, 8, and 10. Digests run overnight at 37° C. for approximately 16 hours on a 1.5% agarose gel. Controls with no cobalt catalyst were always run in parallel. b) 5 mM cocycmmb and 1.19 μg (0.168 mM bp) pBluescript plasmid DNA in 0.1M MOPS buffer at pH 7, 8, and 10 run under identical conditions. 1=Nickase+DNA, 2=DNA, 3=Cobalt complex+DNA at pH 7, 4=pH 7 control, 5=Cobalt complex+DNA at pH 8, 6=pH 8 control, 7=Cobalt complex+DNA at pH 10, 8=pH 10 control Nickase reactions were performed at pH 7 using a 4 hour, 37° C. incubation of 1.18 μg pBluescript plasmid DNA.

Activity of Cocycmmb in Preventing Translation

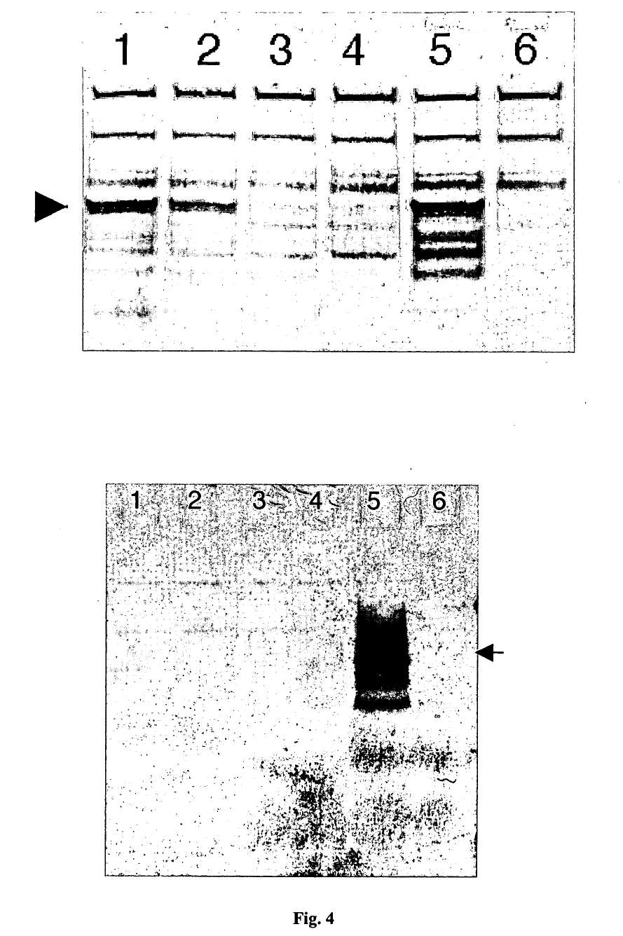

A test of the antiviral potential of these Cocyc-based systems has been performed by demonstrating the ability of Cocycmmb to prevent the translation of RNA into its protein product. As shown in FIG. 4, the absence of the protein band in the Western blot is shown as a function of Cocycmmb concentration and also as a function of time of contact between Cocycmmb and the RNA. Most recently, we have observed significant inhibition of protein production with as little as 0.25 mM at 10 minutes incubation time.

The highly effective suppression of protein production by the translational mechanism demonstrates our concept that these metal-chelator complexes can be used for prevention of viral replication or other biological reproductions that require protein translation. This property is especially central to using these compounds as antiviral, antibacterial clean-up agents, where sequence specificity is not needed.

Activity of Cocycmmb in the Hydrolysis of RNA

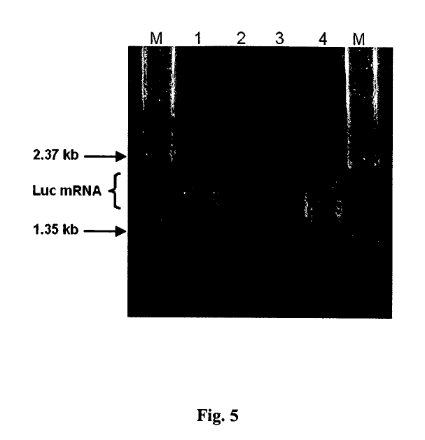

A further demonstration of the gene-silencing ability of these Cocyc-based systems was evidenced by the demonstration of the ability of Cocycmmb to hydrolyze RNA. The direct interaction of Co(III)-cycmmb with luciferase mRNA was analyzed by denaturing agarose gel electrophoresis. mRNA template (2 μg at a final concentration of 0.4 mg/mL) was incubated with varying concentrations (1 to 5 mM) of activated Co(III)-cycmmb for 24 h at 25° C. in 5 μL of HBS. Following the incubation, the reactions were analyzed by electrophoresis on a 2% denaturing agarose gel run for 4 h at 40 V. The gel was stained with RiboGreen (Molecular Probes), visualized by UV illumination, and images were quantified using Scion Image using the control incubation (no Co(III)-cycmmb) as a standard (see FIG. 5).

Reactivity of the Functionalized Gel

It was questioned whether the hydrolytic activity of the cobalt cyclen complex retains its activity when anchored to a solid support, such as a commercially available agarose beads. The bead matrix is derivatized with a nine-atom spacer that terminates in a primary amine functional group (see Scheme 5). The cobalt concentration was determined using ICP-MS and the efficiency of conjugation for the EDC-NHS coupled agarose gel was determined to be 73%. In the absence of EDC and NHS, the efficiency of coupling of the complex to the agarose gel was extremely low (approximately 1.3%), indicating that the conjugation reaction between the Co(III)-cyclenmba complex and the agarose gel was highly specific.

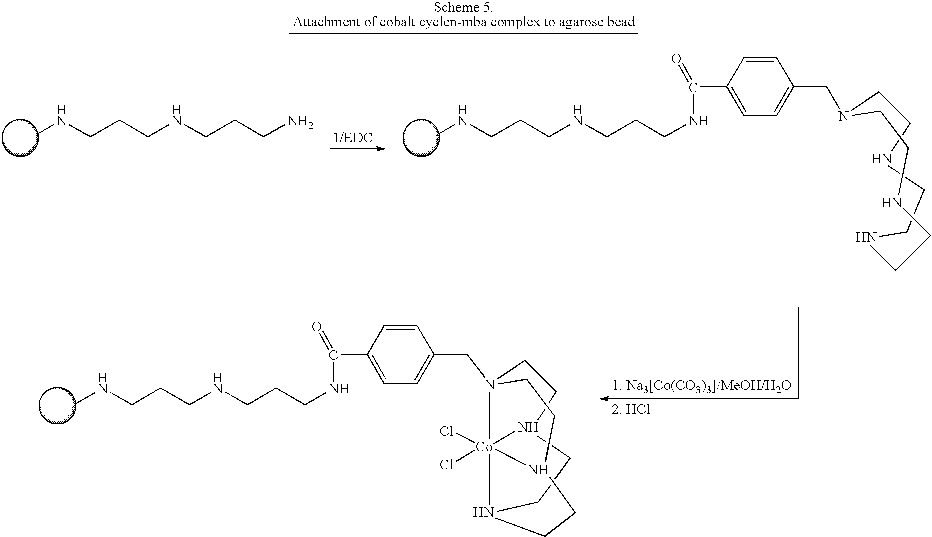

To demonstrate that the catalytic activity of the cyclen complex is retained on grafting to the agarose bead support, the hydrolysis of BNPP was performed at 45° C. in MOPS buffer using the activated, supported catalyst. After overnight stirring, the mixture was filtered to remove the suspended beads and a visible spectrum of the resulting solution was recorded. The spectrum showed complete hydrolysis of BNPP to NPP, confirming retention of activity of the cobalt complex after conjugation to the DADPA gel. Detailed kinetic analysis of the reaction was not performed.

The above description is that of a preferred embodiment of the invention. Various modifications and variations are possible in light of the above teachings. It is therefore to be understood that, within the scope of the appended claims, the invention may be practiced otherwise than as specifically described. Any reference to claim elements in the singular, e.g., using the articles “a,” “an,” “the,” or “said” is not construed as limiting the element to the singular.

What is claimed is:

1. A composition comprising:
a kinetically inert ion coordinated to a macrocyclic chelator or ligand;
a first functional group bonded to said macrocyclic chelator or ligand;
an oligonucleotide; and
a second functional group bonded to said oligonucleotide,
wherein said first functional group covalently bonds to said second functional group.

2. The composition of claim 1 wherein said kinetically inert ion is selected from the group consisting of Co(III), Cr(III), Rh(III), Pt(II), and Ir(III).

3. The composition of claim 1 wherein said macrocyclic chelator is selected from the group consisting of 1,4,7,11-tetraazacyclododecane (cyclen), trpn tris(3-aminopropyl)amine (trpn), and tris(2-aminoethyl)amine (tren).

4. The composition of claim 1 wherein said oligonucleotide is selected from the group consisting of a standard oligonucleotide, an oligonucleotide composed of locked nucleic acids, and a peptide nucleic acid.

5. The composition of claim 1 wherein said first functional group is selected from the group consisting of an amine, a carboxylic acid, a thiol, an isocyanate, an isothiocyanate, a maleimide, and an epoxide.

6. The composition of claim 1 wherein said second functional group is selected from the group consisting of an amine, a carboxylic acid, a thiol, an isocyanate, an isothiocyanate, a maleimide, and an epoxide.

7. The composition of claim 1 wherein said kinetically inert ion is Co(III) and wherein said macrocyclic chelator is selected from the group consisting of 1,4,7,11-tetraazacyclododecane (cyclen), trpn tris(3-aminopropyl)amine (trpn), and tris(2-aminoethyl)amine (tren) and wherein said first functional group is selected from the group consisting of an amine, a carboxylic acid, a thiol, an isocyanate, an isothiocyanate, a maleimide, and an epoxide and wherein said second functional group is selected from the group consisting of an amine, a carboxylic acid, a thiol, an isocyanate, an isothiocyanate, a maleimide, and an epoxide.

8. The composition of claim 1 wherein said kinetically inert ion is Co(III) and wherein said macrocyclic chelator is 1,4,7,11-tetraazacyclododecane (cyclen) and wherein said first and said second functional group is selected from the group consisting of an amine, a carboxylic acid, a thiol, an isocyanate, an isothiocyanate, a maleimide, and an epoxide.

9. A composition comprising:
a Co(III) ion in a macrocyclic chelator bonded to a first functional group having a carboxylic acid moiety; and
an oligonucleotide bonded to a second functional group having a primary amine moiety, wherein said carboxylic acid moiety covalently bonds to said primary amine moiety.

10. The composition of claim 9 wherein said oligonucleotide is selected from the group consisting of a standard oligonucleotide, an oligonucleotide composed of locked nucleic acids, and a peptide nucleic acid.

11. The composition of claim 9 wherein said macrocyclic chelator is selected from the group consisting of 1,4,7,11-tetraazacyclododecane (cyclen), trpn tris(3-aminopropyl)amine (trpn), and tris(2-aminoethyl)amine (tren).

12. A method of making a macrocyclic chelator comprising:
converting Co(II)Cl₂ to a stabilized Co(III) complex;
reacting the Co(III)complex with a cyclen ligand comprising a carboxylic acid moiety through which amine bond formation with a primary amine can be achieved; and
reacting the Co(III) cyclen carboxylic acid complex with a suitable modified oligonucleotide to form a sequence-specific gene silencing moiety.
